(19) United States
(12) Patent Application Publication
(10) Pub. No.: US 2016/0250222 A1
(43) Pub. Date: Sep. 1, 2016

(54) DRUG FOR PREVENTING AND/OR TREATING POLYCYSTIC KIDNEY DISEASE

(71) Applicant: OTSUKA PHARMACEUTICAL CO., LTD., Chiyoda-ku, Tokyo (JP)

(72) Inventors: Hiroyuki FUJIKI, Osaka-shi (JP); Miki AIHARA, Osaka-shi (JP); Katsuji HATTORI, Osaka-shi (JP); Koji OHMOTO, Osaka-shi (JP); Takakuni MATSUDA, Osaka-shi (JP); Daiki KANEKO, Osaka-shi (JP)

(73) Assignee: OTSUKA PHARMACEUTICAL CO., LTD., Tokyo (JP)

(21) Appl. No.: 15/028,450

(22) PCT Filed: Oct. 14, 2014
(86) PCT No.: PCT/JP2014/077763

§ 371 Date: Apr. 11, 2016

(30) Foreign Application Priority Data
Oct. 15, 2013 (JP) 2013-215137

(51) Int. Cl. A61K 31/55; A61K 9/00; A61K 38/10

(52) U.S. Cl. CPC A61K 31/55; A61K 38/10; A61K 9/0019

(57) ABSTRACT

The present invention provides a combined drug for an injectable depot formulation having a superior effect of preventing and/or treating polycystic kidney disease. More specifically, the present invention relates to a drug for preventing and/or treating polycystic kidney disease, which is an injectable depot formulation comprising a particle containing tolvaptan or a prodrug thereof and a somatostatin analogue. The present invention also relates to a method for preventing and/or treating polycystic kidney disease using the drug.

### TECHNICAL FIELD

The present invention relates to a drug for preventing and/or treating polycystic kidney disease (PKD).

### BACKGROUND ART

Polycystic kidney disease is classified into ADPKD (autosomal dominant polycystic kidney disease) and ARPKD (autosomal recessive polycystic kidney disease). In both types of polycystic kidney disease, many cysts develop in the cortex and medulla of the kidney, leading to kidney dysfunction accompanied by atrophy and fibrosis of parenchyma. As the disease progresses, many cysts progressively develop and grow, and the kidney function decreases, leading to end-stage kidney failure requiring dialysis.

In cyst epithelial cells wherein cysts develop from tubular cells, cyclic AMP (cAMP) activates protein kinase A (PKA), and a series of MAP kinase (MAPK) pathways are activated to induce cell proliferation. In the cyst epithelial cells, the expression of vasopressin V2 receptor (V2R) is enhanced and adenylate cyclase activity is elevated, which further increases cAMP levels and accelerates cell proliferation.

Vasopressin V2 receptor (V2R) antagonists have been reported to have complete response in animal models of polycystic kidney disease. Tolvaptan is advancing in clinical trials, and has been approved and is commercially available in Japan (see, for example, Patent Literature 1 and Non-Patent Literature 1, 2, and 3).

Octreotide, which is a somatostatin analogue that suppresses adenylate cyclase activity, is also expected to be useful as an agent for treating ADPKD, and clinical test results therefor have recently been reported (see Non-Patent Literature 4).

### CITATION LIST

### Patent Literature

PTL 1: JP4-154765A

### Non-Patent Literature

NPL 1: Nat. Med., 2003, 9(10):1323-6
NPL 2: J. Am. Soc. Nephrol., 2005, 16:846-851
NPL 3: Clin. J. Am. Soc. Nephrol., 2008, 3:1212-1218
NPL 4: J. Am. Soc. Nephrol., 2010, 21:1052-1061

### SUMMARY OF INVENTION

### Technical Problem

An object of the present invention is to provide an injectable depot formulation having a superior effect of preventing and/or treating polycystic kidney disease.

### Solution to Problem

To attain the above object, the present invention conducted extensive research on an injectable depot formulation comprising a combination of drugs that can significantly increase the effect of preventing and/or treating polycystic kidney disease.

As a result, it was confirmed that the use of an injectable depot formulation comprising a particle containing tolvaptan; and octreotide or lanreotide, which is a somatostatin analogue, can ensure a significant effect of treating polycystic kidney disease (e.g., an effect of suppressing an increase in weight or volume of kidney, an effect of improving kidney function, etc.), compared with the case of administering either drug alone.

Further, it was also confirmed that by combining a particle containing tolvaptan and a somatostatin analogue as in the above combined drug, it is possible to ensure a significant effect of treating polycystic kidney disease even when the individual doses of the tolvaptan and the somatostatin analogue are so low as to be ineffective if either of the drugs is administered alone.

Based on such findings, further research was conducted, and the present invention was completed.

Specifically, the present invention provides the following combined drugs.

Item 1. A drug for preventing and/or treating polycystic kidney disease, wherein the drug is an injectable depot formulation comprising a particle containing tolvaptan or a prodrug thereof, and a somatostatin analogue.
Item 2. The drug for preventing and/or treating polycystic kidney disease according to Item 1, wherein the tolvaptan or a prodrug thereof is crystalline.
Item 3. The drug for preventing and/or treating polycystic kidney disease according to Item 1 or 2, wherein the tolvaptan or a prodrug thereof is an optically active body.
Item 4. The drug for preventing and/or treating polycystic kidney disease according to Item 3, wherein the optically active tolvaptan or a prodrug thereof is tolvaptan consisting essentially of R-tolvaptan or a prodrug thereof, or tolvaptan consisting essentially of S-tolvaptan or a prodrug thereof.
Item 5. The drug for preventing and/or treating polycystic kidney disease according to any one of Items 1 to 4, wherein the content of tolvaptan or a prodrug thereof in the particle containing tolvaptan or a prodrug thereof is 50 to 100 wt %.
Item 6. The drug for preventing and/or treating polycystic kidney disease according to any one of Items 1 to 5, wherein the particle containing tolvaptan or a prodrug thereof has a mean particle size of about 1 to 100 μm.
Item 7. The drug for preventing and/or treating polycystic kidney disease according to any one of Items 1 to 6, wherein the somatostatin analogue is at least one member selected from the group consisting of somatostatin, octreotide, pasireotide, lanreotide, vapreotide, and their salts.
Item 8. The drug for preventing and/or treating polycystic kidney disease according to any one of Items 1 to 7, wherein the somatostatin analogue is a microsphere formulation or a gel formulation.
Item 9. The drug for preventing and/or treating polycystic kidney disease according to any one of Items 1 to 8, wherein the injectable depot formulation comprises a mixture of a particle containing tolvaptan or a prodrug thereof and a somatostatin analogue (mixed drug).
Item 10. The drug for preventing and/or treating polycystic kidney disease according to Item 9, wherein the injectable depot formulation contains a pharmaceutically acceptable carrier for injection.
Item 11. The drug for preventing and/or treating polycystic kidney disease according to Item 9 or 10, wherein the injectable depot formulation contains water for injection.
Item 12. The drug for preventing and/or treating polycystic kidney disease according to Item 11, wherein the injectable depot formulation is in the form of an aqueous suspension, and the aqueous suspension contains tolvaptan or a prodrug thereof in an amount of 50 mg/mL to 500 mg/mL.
Item 13. The drug for preventing and/or treating polycystic kidney disease according to any one of Items 9 to 12, wherein the injectable depot formulation is administered subcutaneously or intramuscularly.
Item 14. The drug for preventing and/or treating polycystic kidney disease according to any one of Items 1 to 13, wherein the injectable depot formulation is administered once during a two-week or more interval.
Item 15. The drug for preventing and/or treating polycystic kidney disease according to any one of Items 1 to 8, wherein the injectable depot formulation comprises a combination of Injectable Depot Formulation A comprising a particle containing tolvaptan or a prodrug thereof, and Injectable Depot Formulation B comprising a somatostatin analogue, and Formulation A and Formulation B are administered in combination (drug for combination use).
Item 16. The drug for preventing and/or treating polycystic kidney disease according to Item 15, wherein Injectable Depot Formulation A contains a pharmaceutically acceptable carrier for injection.
Item 17. The drug for preventing and/or treating polycystic kidney disease according to Item 15 or 16, wherein Injectable Depot Formulation B contains a pharmaceutically acceptable carrier for injection.
Item 18. The drug for preventing and/or treating polycystic kidney disease according to any one of Items 15 to 17, wherein Injectable Depot Formulations A and B contain water for injection.
Item 19. The drug for preventing and/or treating polycystic kidney disease according to any one of Items 15 to 18, wherein Injectable Depot Formulation A is in the form of an aqueous suspension, and the aqueous suspension contains tolvaptan or a prodrug thereof in an amount of 50 mg/mL to 500 mg/mL.
Item 20. The drug for preventing and/or treating polycystic kidney disease according to any one of Items 15 to 19, wherein Injectable Depot Formulation B is in the form of an aqueous suspension or a gel.
Item 21. The drug for preventing and/or treating polycystic kidney disease according to any one of Items 15 to 20, wherein each of Injectable Depot Formulations A and B is administered subcutaneously or intramuscularly.
Item 22. The drug for preventing and/or treating polycystic kidney disease according to any one of Items 1 to 21, wherein the tolvaptan or a prodrug thereof is tolvaptan.
Item 23. A kit (or a package) for preventing and/or treating polycystic kidney disease, comprising a combination of a container having Injectable Depot Formulation A comprising a particle containing tolvaptan or a prodrug thereof; and a container having Injectable Depot Formulation B comprising a somatostatin analogue.
Item 24. A method for preventing and/or treating polycystic kidney disease, comprising administering an injectable depot formulation comprising a particle containing tolvaptan or a prodrug thereof and a somatostatin analogue to a patient in need of prevention and/or treatment of polycystic kidney disease.
Item 25. The method for preventing and/or treating polycystic kidney disease according to Item 24, comprising administering, either at the same time or with a time difference, Injectable Depot Formulation A comprising a particle containing tolvaptan or a prodrug thereof, and Injectable Depot Formulation B comprising a somatostatin analogue to a patient in need of prevention and/or treatment of polycystic kidney disease.
Item 26. Use of an injectable depot formulation comprising a particle containing tolvaptan or a prodrug thereof and a somatostatin analogue for the production of a drug for preventing and/or treating polycystic kidney disease.
Item 27. Use of a combination of Injectable Depot Formulation A comprising a particle containing tolvaptan or a prodrug thereof and Injectable Depot Formulation B comprising a somatostatin analogue for the production of a drug for preventing and/or treating polycystic kidney disease.
Item 28. An injectable depot formulation comprising a particle containing tolvaptan or a prodrug thereof and a somatostatin analogue for use as a drug for preventing and/or treating polycystic kidney disease.
Item 29. A combination of Injectable Depot Formulation A comprising a particle containing tolvaptan or a prodrug thereof and Injectable Depot Formulation B comprising a somatostatin analogue for use as a drug for preventing and/or treating polycystic kidney disease.
Item 30. An injectable depot formulation comprising a particle containing tolvaptan or a prodrug thereof and a somatostatin analogue for use in preventing and/or treating polycystic kidney disease.
Item 31. A combination of Injectable Depot Formulation A comprising a particle containing tolvaptan or a prodrug thereof and Injectable Depot Formulation B comprising a somatostatin analogue for use in preventing and/or treating polycystic kidney disease.
Item 32. Injectable Depot Formulation A comprising a particle containing tolvaptan or a prodrug thereof to be used with Injectable Depot Formulation B comprising a somatostatin analogue.
Item 33. Injectable Depot Formulation B comprising a somatostatin analogue to be used with Injectable Depot Formulation A comprising a particle containing tolvaptan or a prodrug thereof.
Item 34. A drug for inhibiting increase in weight or volume of kidney of a polycystic kidney disease patient, wherein the drug is an injectable depot formulation comprising a particle containing tolvaptan or a prodrug thereof and a somatostatin analogue.
Item 35. A drug for inhibiting increase in weight or volume of kidney of a polycystic kidney disease patient, wherein the drug comprises a combination of Injectable Depot Formulation A comprising a particle containing tolvaptan or a prodrug thereof and Injectable Depot Formulation B comprising a somatostatin analogue.

### Advantageous Effects of Invention

The injectable depot formulation comprising a particle containing tolvaptan or a prodrug thereof and a somatostatin analogue of the present invention makes it possible to significantly increase an effect of preventing and/or treating polycystic kidney disease while improving the quality of life (QOL) and the adherence of the patient.

The formulation of the present invention encompasses both a mixed drug (mixture) and a drug for combination use. For example, the formulation of the present invention encompasses an injectable depot formulation (mixture) obtained by mixing a particle containing tolvaptan or a prodrug thereof with a somatostatin analogue; and a drug (drug for combination use) that includes Injectable Depot Formulation A comprising a particle containing tolvaptan or a prodrug thereof and Injectable Depot Formulation B comprising a somatostatin analogue, and enables combined administration of these separate formulations.

Since tolvaptan disappears relatively rapidly when orally administered, a high dose of tolvaptan has been required to be orally administered twice a day to steadily suppress the action of vasopressin. Further, there have been cases where oral administration of tolvaptan causes excessive diuretic effect due to the high maximum blood concentration of tolvaptan; reduction in vasopressin antagonism due to the rapid disappearance of tolvaptan from the blood; and the like. This may result in frequent urination, in particular, nocturia. There is thus room for further improvement in the quality of life (QOL) of patients. Further, since patients must take a drug for their lifetimes in the treatment of polycystic kidney disease, there has been a demand to reduce the frequency of administration of tolvaptan from the viewpoint of quality of life (QOL) and adherence of patients.

The drug for preventing and/or treating polycystic kidney disease of the present invention comprises a particle containing tolvaptan or a prodrug thereof, and thus maintains a therapeutically effective blood concentration of tolvaptan for a long period of time. Further, the use of a combination of tolvaptan and a somatostatin analogue can provide a remarkable therapeutic effect on polycystic kidney disease, even when the individual doses of the tolvaptan and the somatostatin analogue are so low as to be ineffective if an injectable depot formulation comprising either of a tolvaptan or a somatostatin analogue is administered alone. Additionally, by the combined administration of these drugs, the increase in urine output (such as pollakiuria) can be solved compared with administration of an injectable depot formulation comprising tolvaptan alone or oral administration of tolvaptan alone, thereby maintaining both Quality of Life (QOL) and adherence of polycystic kidney disease patients.

In particular, use of crystalline tolvaptan provides an excellent effect such that stable blood concentration of tolvaptan can be maintained by a single administration of crystalline tolvaptan for a long period of time. Further, use of an optically active tolvaptan provides an excellent effect that the drug administration amount for ensuring the therapeutically effective tolvaptan concentration can be reduced compared with the case using a racemic body if a desirable optically active body is selected. Further, use of a crystalline and optically active tolvaptan (in particular, R-tolvaptan or S-tolvaptan) provides an excellent effect such that the absorption of a crystalline and optically active tolvaptan is faster than a crystalline racemic body, and thus a higher blood concentration of tolvaptan can be ensured; and such that, since crystalline transition does not easily occur, the therapeutically effective tolvaptan concentration can be maintained at a constant level for four weeks or more by a single administration. Among crystalline optically active tolvaptans, crystalline S-tolvaptan is most preferable in terms of its high metabolic stability in humans.

### BRIEF DESCRIPTION OF DRAWINGS

FIG. 1 shows a polarizing microscope image of an injectable depot formulation comprising a crystalline S-tolvaptan particle produced in Production Example 2.

FIG. 2 shows a polarizing microscope image of an octreotide acetate sustained-release formulation (Sandostatin LAR (registered trademark; the same hereinafter)).

FIG. 3 shows a polarizing microscope image of an injectable depot formulation comprising a crystalline R-tolvaptan particle produced in Production Example 3.

FIG. 4 shows a polarizing microscope image of an injectable depot formulation (mixed drug) comprising a crystalline S-tolvaptan particle and an octreotide acetate sustained-release formulation (Sandostatin LAR) produced in Production Example 4.

FIG. 5 shows a polarizing microscope image of an injectable depot formulation (mixed drug) comprising a crystalline R-tolvaptan particle and an octreotide acetate sustained-release formulation (Sandostatin LAR) produced in Production Example 5.

### DESCRIPTION OF EMBODIMENTS

The drug for preventing and/or treating polycystic kidney disease of the present invention is an injectable depot formulation comprising a particle containing tolvaptan or a prodrug thereof, and a somatostatin analogue. More specifically, the drug for preventing and/or treating polycystic kidney disease of the present invention is a combination of a particle containing tolvaptan or a prodrug thereof, and a somatostatin analogue. The drug of the present invention encompasses a mixed drug (mixed drug) obtained by mixing the particle containing tolvaptan or a prodrug thereof, and a somatostatin analogue; and a formulation (drug for combination use) that enables combined administration of two separate formulations, i.e., Injectable Depot Formulation A containing the particle containing tolvaptan or a prodrug thereof, and Injectable Depot Formulation B comprising a somatostatin analogue.

### 1. Particle Containing Tolvaptan or Prodrug Thereof

The drug for preventing and/or treating polycystic kidney disease of the present invention is an injectable depot formulation comprising a particle containing tolvaptan or a prodrug thereof. With this drug, the therapeutically effective blood concentration of tolvaptan can be maintained for a long period of time.

Tolvaptan is the common name for 7-chloro-5-hydroxy-1-[2-methyl-4-(2-methylbenzoylamino)benzoyl]-2,3,4,5-tetrahydro-1H-benzoazepine, represented by Formula (I).

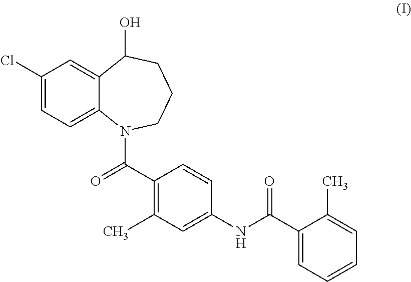 [SMILES: Cc1ccccc1C(=O)Nc1ccc(C(=O)N2CCCC(O)c3cc(Cl)ccc32)c(C)c1]

Tolvaptan contains a hydroxy-bonded carbon atom as an asymmetric carbon, as shown in Formula (I). Therefore, tolvaptan has a pair of enantiomers (R- and S-enantiomers) based on the asymmetric carbon. The term “tolvaptan” is used to include R-tolvaptan, S-tolvaptan, and a mixture of the two enantiomers in any proportion. Tolvaptan is typically an optically active body (in particular, R-tolvaptan or S-tolvaptan that is optical isomer or an enantiomer), or a racemic body.

Tolvaptan may be a prodrug. A prodrug is a compound obtained by modifying an active compound (tolvaptan) in consideration of improved solubility in water, improved stabilization, improved bioavailability, etc. The later-described prodrug has a pair of enantiomers (R-enantiomer and S-enantiomer) based on the asymmetric carbon that is derived from the hydroxy-bonded carbon atom in Formula (I) above.

An example of a prodrug is a compound produced by phosphorylation of the hydroxy group of tolvaptan. Specific examples thereof include a benzazepine compound represented by Formula (Ia) below or a salt thereof, which is disclosed in JP2009-521397A.

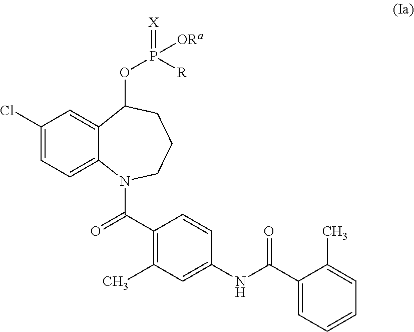

wherein R is a hydrogen atom, a hydroxy group optionally having a protecting group, a mercapto group optionally having a protecting group, or an amino group optionally having one or two protecting groups; R^{a} is a hydrogen atom or a hydroxy-protecting group; and X is an oxygen atom or a sulfur atom.

In Formula (Ia), there is no particular limitation on the “protecting group” for the hydroxy group optionally having a protecting group, the mercapto group optionally having a protecting group, or the amino group optionally having one or two protecting groups, represented by R. Typical examples of protecting groups include lower alkyl groups (for example, C₁₋₆ alkyl groups, such as methyl and ethyl), phenyl-lower alkyl groups (for example, phenyl-C₁₋₆ alkyl groups, such as benzyl and phenethyl), lower alkoxycarbonyl groups (for example, C₁₋₆ alkoxycarbonyl groups, such as methoxycarbonyl, ethoxycarbonyl, and tert-butoxycarbonyl), and the like.

Examples of the hydroxy protecting group represented by R^{a} in Formula (Ia) are those mentioned above as examples of the “protecting group” included in R.

Other examples of prodrugs include a compound obtained by acylating the hydroxy group of tolvaptan. Specific examples thereof include a benzazepine compound represented by the following formula (Ib) or a salt thereof, which are disclosed in WO2009/001968 (JP2010-531293A).

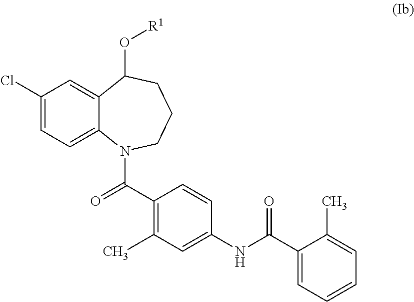

wherein R¹ is a group of (1-1) to (1-7) below:

(1-1) a —CO—(CH₂)ₙ—COR² group

wherein n is an integer of 1 to 4, R² is (2-1) a hydroxy group; (2-2) a lower alkoxy group optionally substituted with a hydroxy group, a lower alkanoyl group, a lower alkanoyloxy group, a lower alkoxycarbonyloxy group, a cycloalkyloxycarbonyloxy group, or 5-methyl-2-oxo-1,3-dioxol-4-yl; or (2-3) an amino group optionally substituted with a hydroxy-lower alkyl group;

(1-2) a —CO—(CH₂)ₘ—NR³R⁴ group

wherein m is an integer of 0 to 4, R³ is a hydrogen atom or a lower alkyl group, R⁴ is (4-1) a hydrogen atom; (4-2) a lower alkyl group optionally substituted with a halogen atom, a lower alkylamino group, a lower alkoxycarbonyl group, or 5-methyl-2-oxo-1,3-dioxol-4-yl; or (4-3) a lower alkoxycarbonyl group optionally substituted with a halogen atom, a lower alkanoyloxy group, or 5-methyl-2-oxo-1,3-dioxol-4-yl, R³ and R⁴ may form a 5- or 6-membered saturated heterocyclic ring by bonding R³ and R⁴ to each other, together with the nitrogen atom to which R³ and R⁴ bond, directly or via a nitrogen atom or oxygen atom, the heterocyclic ring being optionally substituted with (4-4) a lower alkyl group (the lower alkyl group being optionally substituted with a hydroxy-lower alkoxy group); (4-5) a lower alkoxycarbonyl group; (4-6) an alkylcarbonyl group (the alkyl group being optionally substituted with a carboxyl group or a lower alkoxycarbonyl group); (4-7) an arylcarbonyl group; or (4-8) a furylcarbonyl group;

(1-3) a —CO—(CH₂)ₚ—O—CO—NR⁵R⁶ group

wherein p is an integer of 1 to 4, R⁵ is a lower alkyl group, and R⁶ is a lower alkoxycarbonyl-lower alkyl group;

(1-4) a —CO—(CH₂)_{q}—X—R⁷ group

wherein q is an integer of 1 to 4, X is an oxygen atom, a sulfur atom, or a sulfonyl group, and R⁷ is a carboxy-lower alkyl group, or a lower alkoxycarbonyl-lower alkyl group;

(1-5) a —CO—R⁸ group

wherein R⁸ is (8-1) an alkyl group optionally substituted with a halogen atom, a lower alkanoyloxy group, or a phenyl group (the phenyl group being substituted with a dihydroxyphosphoryloxy group in which either or both of the hydroxy groups are optionally substituted with benzyl groups, and a lower alkyl group), (8-2) a lower alkoxy group substituted with a halogen atom, a lower alkanoyloxy group, or a dihydroxyphosphoryloxy group, (8-3) a pyridyl group, or (8-4) a lower alkoxyphenyl group;

(1-6) a lower alkyl group substituted with a group selected from the group consisting of lower alkylthio groups, a dihydroxyphosphoryloxy group, and lower alkanoyloxy groups; and

(1-7) an amino acid or peptide residue optionally protected with one or more protecting groups.

In Formula (Ib), the term “lower” refers to “C₁₋₆,” unless otherwise specified.

Examples of lower alkanoyl groups include straight or branched C₂₋₆ alkanoyl groups, such as acetyl, n-propionyl, n-butyryl, isobutyryl, n-pentanoyl, tert-butylcarbonyl, and n-hexanoyl.

Examples of lower alkanoyloxy groups include straight or branched C₂₋₆ alkanoyloxy groups, such as acetyloxy, n-propionyloxy, n-butyryloxy, isobutyryloxy, n-pentanoyloxy, tert-butylcarbonyloxy, and n-hexanoyloxy.

Examples of lower alkoxycarbonyloxy groups include alkoxycarbonyloxy groups in which the alkoxy moiety is a straight or branched C₁₋₆ alkoxy group, such as methoxycarbonyloxy, ethoxycarbonyloxy, n-propoxycarbonyloxy, isopropoxycarbonyloxy, n-buthoxycarbonyloxy, isobuthoxycarbonyloxy, tert-buthoxycarbonyloxy, sec-buthoxycarbonyloxy, n-pentyloxycarbonyloxy, neopentyloxycarbonyloxy, n-hexyloxycarbonyloxy, isohexyloxycarbonyloxy, and 3-methyl pentyloxycarbonyloxy.

Examples of cycloalkyloxycarbonyloxy groups include cycloalkyloxycarbonyloxy groups in which the cycloalkyl moiety is a C₃₋₈ cycloalkyl group, such as cyclopropyloxycarbonyloxy, cyclobutyloxycarbonyloxy, cyclopentyloxycarbonyloxy, cyclohexyloxycarbonyloxy, cycloheptyloxycarbonyloxy, and cyclooctyloxycarbonyloxy.

Examples of cycloalkylcarbonyl groups include cycloalkylcarbonyl groups in which the cycloalkyl moiety is a C₃₋₈ cycloalkyl group, such as cyclopropylcarbonyl, cyclobutylcarbonyl, cyclopentylcarbonyl, cyclohexylcarbonyl, cycloheptylcarbonyl, and cyclooctylcarbonyl.

Examples of lower alkoxy groups include straight or branched C₁₋₆ alkoxy groups, such as methoxy, ethoxy, n-propoxy, isopropoxy, n-butoxy, isobutoxy, tert-butoxy, sec-butoxy, n-pentyloxy, isopentyloxy, neopentyloxy, n-hexyloxy, isohexyloxy, and 3-methylpentyloxy.

Examples of hydroxy-lower alkyl groups include straight or branched C₁₋₆ alkyl groups having one to three hydroxy groups, such as hydroxymethyl, 2-hydroxyethyl, 1-hydroxyethyl, 3-hydroxypropyl, 2,3-dihydroxypropyl, 4-hydroxybutyl, 3,4-dihydroxybutyl, 1,1-dimethyl-2-hydroxyethyl, 5-hydroxypentyl, 6-hydroxyhexyl, 3,3-dimethyl-3-hydroxypropyl, 2-methyl-3-hydroxypropyl, and 2,3,4-trihydroxybutyl.

Examples of alkyl groups include straight or branched C₁₋₁₀ alkyl groups such as methyl, ethyl, n-propyl, isopropyl, n-butyl, isobutyl, tert-butyl, sec-butyl, n-pentyl, isopentyl, neopentyl, n-hexyl, isohexyl, and 3-methylpentyl.

Examples of lower alkyl groups include straight or branched C₁₋₆ alkyl groups, such as methyl, ethyl, n-propyl, isopropyl, n-butyl, isobutyl, tert-butyl, sec-butyl, n-pentyl, isopentyl, neopentyl, n-hexyl, isohexyl, and 3-methylpentyl.

Examples of halogen atoms include fluorine, chlorine, bromine, and iodine.

Examples of lower alkylamino groups include amino groups substituted with one to two straight or branched C₁₋₆ alkyl groups, such as methylamino, ethylamino, n-propylamino, isopropylamino, n-butylamino, tert-butylamino, n-pentylamino, n-hexylamino, dimethylamino, diethylamino, di-n-propylamino, di-n-butylamino, di-n-pentylamino, di-n-hexylamino, N-methyl-N-ethylamino, N-ethyl-N-n-propylamino, N-methyl-N-n-butylamino, and N-methyl-N-n-hexylamino.

Examples of lower alkoxycarbonyl groups include alkoxycarbonyl groups in which the alkoxy moiety is a straight or branched C₁₋₆ alkoxy group, such as methoxycarbonyl, ethoxycarbonyl, n-propoxycarbonyl, isopropoxycarbonyl, n-butoxycarbonyl, isobutoxycarbonyl, tert-butoxycarbonyl, sec-butoxycarbonyl, n-pentyloxycarbonyl, neopentyloxycarbonyl, n-hexyloxycarbonyl, isohexyloxycarbonyl, and 3-methylpentyloxycarbonyl.

Examples of 5- or 6-membered saturated heterocyclic rings formed by bonding R³ and R⁴ to each other, together with the nitrogen atom to which R³ and R⁴ bond, directly or via a nitrogen atom or oxygen atom include pyrrolidine, imidazolidine, piperazine, piperidine, and morpholine.

Examples of hydroxy-lower alkoxy groups include hydroxyalkoxy groups that have one or two hydroxy groups, the alkoxy moiety being a straight or branched C₁₋₆ alkoxy group, such as hydroxymethoxy, 2-hydroxyethoxy, 1-hydroxyethoxy, 3-hydroxypropoxy, 4-hydroxybutoxy, 5-hydroxypentyloxy, 6-hydroxyhexyloxy, 1,1-dimethyl-2-hydroxyethoxy, and 2-methyl-3-hydroxypropoxy.

Examples of alkylcarbonyl groups include alkylcarbonyl groups in which the alkyl moiety is a straight or branched C₁₋₂₀ alkyl group, such as methylcarbonyl, ethylcarbonyl, n-propylcarbonyl, isopropylcarbonyl, n-butylcarbonyl, isobutylcarbonyl, tert-butylcarbonyl, sec-butylcarbonyl, n-pentylcarbonyl, isopentylcarbonyl, neopentylcarbonyl, n-hexylcarbonyl, isohexylcarbonyl, 3-methylpentylcarbonyl, n-heptylcarbonyl, n-octylcarbonyl, n-nonylcarbonyl, n-decylcarbonyl, n-undecylcarbonyl, n-dodecylcarbonyl, n-tridecylcarbonyl, n-tetradecylcarbonyl, n-pentadecylcarbonyl, n-hexadecylcarbonyl, n-heptadecylcarbonyl, n-octadecylcarbonyl, n-nonadecylcarbonyl, and n-icosylcarbonyl.

Examples of arylcarbonyl groups include phenylcarbonyl and (1- or 2-)naphthylcarbonyl.

Examples of furylcarbonyl groups include (2- or 3-) furylcarbonyl.

Examples of lower alkoxycarbonyl-lower alkyl groups include alkoxycarbonylalkyl groups in which the alkoxy moiety is a straight or branched C₁₋₆ alkoxy group, and the alkyl moiety is a straight or branched C₁₋₆ alkyl group, such as methoxycarbonylmethyl, ethoxycarbonylmethyl, 2-methoxycarbonylethyl, 2-ethoxycarbonylethyl, 1-ethoxycarbonylethyl, 3-methoxycarbonylpropyl, 3-ethoxycarbonylpropyl, 4-ethoxycarbonylbutyl, 5-isopropoxycarbonyl pentyl, 6-n-propoxycarbonylhexyl, 1,1-dimethyl-2-n-butoxycarbonylethyl, 2-methyl-3-tert-butoxycarbonylpropyl, 2-n-pentyloxycarbonylethyl, and n-hexyloxycarbonylmethyl.

Examples of carboxy-lower alkyl groups include carboxyalkyl groups in which the alkyl moiety is a straight or branched C₁₋₆ alkyl group, such as carboxymethyl, 2-carboxyethyl, 1-carboxyethyl, 3-carboxypropyl, 4-carboxybutyl, 5-carboxypentyl, 6-carboxyhexyl, 1,1-dimethyl-2-carboxyethyl, and 2-methyl-3-carboxypropyl.

Examples of lower alkoxyphenyl groups include alkoxyphenyl groups in which the alkoxy moiety is a straight or branched alkoxy group, such as methoxyphenyl, ethoxyphenyl, n-propoxyphenyl, isopropoxyphenyl, n-butoxyphenyl, isobutoxyphenyl, tert-butoxyphenyl, sec-butoxyphenyl, n-pentyloxyphenyl, isopentyloxyphenyl, neopentyloxyphenyl, n-hexyloxyphenyl, isohexyloxyphenyl, and 3-methylpentyloxyphenyl.

Examples of lower alkylthio groups include straight or branched alkylthio groups, such as methylthio, ethylthio, n-propylthio, isopropylthio, n-butylthio, tert-butylthio, n-pentylthio, and n-hexylthio.

Examples of amino acid or peptide residues include amino acid residues such as alanyl, phenylalanyl, sarcosyl, valyl, leucyl, isoleucyl, prolyl, N-ethylglycyl, N-propylglycyl, N-isopropylglycyl, N-butylglycyl, N-tert-butylglycyl, N-pentylglycyl, N-hexylglycyl, N,N-diethylglycyl, dipropylglycyl, N,N-dibutylglycyl, N,N-dipentylglycyl, dihexylglycyl, N-methyl-N-ethylglycyl, N-methyl-N-propylglycyl, N-methyl-N-butylglycyl, N-methyl-N-pentylglycyl, and N-methyl-N-hexylglycyl; and peptide residues such as sarcosyl-glycyl, glycyl-glycyl, glycyl-sarcosyl, sarcosyl-sarcosyl, alanyl-glycyl, phenylalanyl-glycyl, phenylalanyl-phenylalanyl, glycyl-glycyl-glycyl, N-ethylglycyl-glycyl, N-propylglycyl-glycyl, dimethylglycyl-glycyl, N,N-diethylglycyl-glycyl, N-methyl-N-ethylglycyl-glycyl, sarcosyl-glycyl-glycyl, N-ethylglycyl-glycyl-glycyl, and N,N-dimethylglycyl-glycyl-glycyl.

Examples of protecting groups for amino acids or peptides include those usually used to protect amino groups of amino acids or peptides, such as tert-butoxycarbonyl, benzyloxycarbonyl, fluorenylmethoxycarbonyl, and acetyl.

A racemic tolvaptan is an equal mixture of the two enantiomers, R-tolvaptan and S-tolvaptan, derived from the hydroxy-bonded asymmetric carbon in the compound represented by Formula (I) above. A prodrug of racemic tolvaptan is a compound obtained by modifying the hydroxy group of the racemic tolvaptan (including the compound represented by General Formula (Ia) or General Formula (Ib)).

An optically active tolvaptan or a prodrug thereof means tolvaptan consisting essentially of R-tolvaptan or a prodrug thereof, or tolvaptan consisting essentially of S-tolvaptan or a prodrug thereof.

The tolvaptan consisting essentially of R-tolvaptan or a prodrug thereof is tolvaptan consisting mainly of R-tolvaptan or a prodrug thereof. However, this tolvaptan or a prodrug thereof may include S-tolvaptan, which is an enantiomer of R-tolvaptan, insofar as the effects of the present invention are ensured. More specifically, the optical purity (enantiomeric excess: ee) of R-tolvaptan or a prodrug thereof is generally not less than 80% ee, preferably not less than 90% ee, more preferably not less than 95% ee, further preferably not less than 99% ee, and particularly preferably 100% ee.

Further, the tolvaptan consisting essentially of S-tolvaptan or a prodrug thereof is tolvaptan consisting mainly of S-tolvaptan or a prodrug thereof. However, this tolvaptan or a prodrug thereof may include R-tolvaptan, which is an enantiomer of S-tolvaptan, insofar as the effects of the present invention are ensured. More specifically, the optical purity (enantiomeric excess; ee) of S-tolvaptan or a prodrug thereof is generally not less than 80% ee, preferably not less than 90% ee, more preferably not less than 95% ee, further preferably not less than 99% ee, and particularly preferably 100% ee.

Such optically active tolvaptans may be produced, for example, according to the disclosure of Heterocycles, 54(1), 2001, pp. 131-138; Heterocycles, 56, 2002, pp. 123-128; Tetrahedron: Asymmetry 21, (2010) 2390-2393. The prodrugs of the optically active tolvaptan may be produced, for example, according to the disclosures of JP2009-521397A and WO2009/001968(JP2010-531293A).

Tolvaptan or a prodrug thereof encompasses an anhydride, a solvate (e.g., a hydrate, an alcoholate, etc.), and co-crystals. Further, tolvaptan or a prodrug thereof may encompass a compound in which one or more atoms in the molecule of tolvaptan or a prodrug thereof are replaced by one or more isotopes thereof. Examples of such isotopes include deuterium (²H), tritium (³H), ¹³C, ¹⁴N, ¹⁸O, and the like.

Tolvaptan or a prodrug thereof may be crystalline or amorphous.

The crystalline tolvaptan or a crystalline prodrug of tolvaptan (hereinafter referred to as crystalline tolvaptan or a prodrug thereof) means that the content of crystalline tolvaptan or a prodrug thereof in the whole tolvaptan or a prodrug thereof in the particle is not less than 90 wt %, preferably not less than 95 wt %, more preferably not less than 97 wt %, and particular preferably means that no amorphous tolvaptan or a prodrug thereof is detected.

The amorphous tolvaptan or an amorphous prodrug of tolvaptan (hereinafter referred to as amorphous tolvaptan or a prodrug thereof) means that the content of crystalline tolvaptan or a prodrug thereof in the whole tolvaptan or a prodrug thereof in the particle is less than 10 wt %, preferably less than 5 wt %, more preferably less than 3 wt %, and particular preferably means that no crystalline tolvaptan or a prodrug thereof is detected.

The content of crystalline tolvaptan or a prodrug thereof in the whole tolvaptan or a prodrug thereof in the particle can be determined by measuring the particle using an analyzer such as an X-ray diffractometer, differential scanning calorimeter (DSC), near-infrared (NIR) spectrometer, microcalorimeter, Raman spectrometer, or terahertz spectrometer.

The particle containing tolvaptan or a prodrug thereof, which is the active ingredient, encompasses a particle consisting essentially of tolvaptan or a prodrug thereof (including a particle consisting of tolvaptan or a prodrug thereof), and a particle consisting of tolvaptan or a prodrug thereof and other ingredients.

Examples of other ingredients include additives for controlling the release rate of tolvaptan or a prodrug thereof from the particle, such as a water-soluble polymer and/or a biodegradable polymer.

Examples of water-soluble polymers include polyvinylpyrrolidone (PVP), hydroxypropyl cellulose (HPC), hydroxypropyl methylcellulose phthalate (HPMCP), hydroxypropyl methylcellulose (HPMC), methacrylic acid copolymer L, methylcellulose (MC), and the like. Preferable water-soluble polymers are hydroxypropyl cellulose (HPC), polyvinylpyrrolidone (PVP), and hydroxypropyl methylcellulose phthalate (HPMCP).

Examples of biodegradable polymers include polylactic acids, polyglycolic acids, polycaprolactones, polycarbonates, polyester amides, polyanhydrides, polyamino acids, polyorthoesters, polycyanoacrylates, poly(p-dioxanone)s, poly(alkylene oxalate)s, biodegradable polyurethanes, mixtures thereof, and copolymers thereof. If the polymers contain asymmetric carbon atoms, monomers constituting the polymers may be any of D-form, L-form, or DL-form. L-form is preferable. Preferable biodegradable polymers are polylactic acids and polylactic acid-polyglycolic acid copolymers.

The weight ratio of the tolvaptan or a prodrug thereof to the water-soluble polymer and/or the biodegradable polymer in the particle is generally 1:0 to 1:4, further 4:1 to 1:4, furthermore 4:1 to 1:2, and particularly 2:1 to 1:1. The weight ratio is particularly preferably 1:0.

The content of tolvaptan or a prodrug thereof in the particle is generally 50 to 100 wt %, more preferably 70 to 100 wt %, further preferably 90 to 100 wt %, particularly preferably 100 wt %.

The mean particle size of the particle containing tolvaptan or a prodrug thereof is generally 1 to 100 μm, preferably 1 to 60 μm, more preferably 2 to 50 μm. The mean particle size of the particle is a volume mean diameter, and can be determined using a laser diffraction particle size distribution meter.

The particle containing amorphous tolvaptan or a prodrug thereof is generally prepared by dissolving tolvaptan or a prodrug thereof together with, if necessary, a water-soluble polymer and/or a biodegradable polymer in an organic solvent, and then spray-drying the mixture. Thus, the particle generally has a spherical shape. The mean particle size of the particle can be set within a desired range (1 to 100 μm, preferably 2 to 60 μm, more preferably 4 to 50 μm) by suitably changing the conditions of the spray-drying method.

The particle containing crystalline tolvaptan or a prodrug thereof is prepared, for example, by recrystallizing racemic tolvaptan or a prodrug thereof, or an optically active tolvaptan or a prodrug thereof. The obtained crystal is processed into a particle having a desired mean particle size by hitherto-known pulverization method, preferably wet pulverization method. The mean particle size of the particle may be set to a desired range (1 to 100 μm, preferably 1 to 30 μm, more preferably 1 to 10 μm) by suitably changing the pulverization conditions.

The particle containing crystalline or amorphous tolvaptan or a prodrug thereof may be a microsphere, or a microcapsule particle. The particle may be prepared, for example, by in-water drying, spray drying, in-liquid drying, solvent diffusion, or the like using tolvaptan or a prodrug thereof, and, as necessary, a water-soluble polymer and/or biodegradable polymer.

In the present invention, the crystalline tolvaptan or a prodrug thereof is preferable, and crystalline tolvaptan is more preferable. This is because, by using crystalline tolvaptan, the high blood concentration immediately after administration (initial burst) can be suppressed, compared with amorphous tolvaptan; thereby, the blood concentration of tolvaptan can be maintained for a long period of time with no significant variation.

In the present invention, an optically active tolvaptan or a prodrug thereof is preferable, and an optically active tolvaptan is more preferable. This is because selecting a preferable optically active body results in a superior effect such that the drug administration amount for maintaining the therapeutically effective blood concentration of drug can be reduced, compared with racemic tolvaptan. Further, since an optically active body is not likely to easily cause crystallization from amorphia and crystal transition, the use thereof is also advantageous in that it causes little variation in elution rate or blood concentration. Among the optically active tolvaptans, tolvaptan consisting essentially of S-tolvaptan is more preferable in terms of its high metabolic stability in humans, and pure S-tolvaptan is particularly preferable. Crystalline pure S-tolvaptan is most preferable.

In the present invention, a crystalline optically active tolvaptan or a prodrug thereof is preferable, and a crystalline optically active tolvaptan is more preferable. This is because the use of the crystalline optically active body provides an additional superior effect such that, since the absorption rate of a crystalline optically active body is faster than that of a crystalline racemic body, it is possible to ensure a high blood concentration of tolvaptan; and because the crystal transition does not easily occur, the therapeutically effective blood concentration can be maintained at a constant level for four weeks or more by a single administration. Among crystalline optically active bodies, tolvaptan consisting essentially of S-tolvaptan is more preferable, and pure S-tolvaptan is particularly preferable.

In this specification, tolvaptan and a prodrug thereof (in particular, the compound represented by Formula (Ia) and/or the compound represented by Formula (Ib)) may be used individually, or in combination. In the present invention, tolvaptan is preferable.

### 2. Somatostatin Analogue

The term “somatostatin analogue” refers to somatostatin, and a compound having a somatostatin-like activity having an amino acid sequence (Phe-Trp-Lys-Thr) essential for the physiological activity of somatostatin; or salts thereof. Specific examples include somatostatin, octreotide, pasireotide, lanreotide, and vapreotide. Such compounds can be used singly, or in a combination of two or more. Octreotide and lanreotide are preferable.

Octreotide refers to a cyclic polypeptide represented by Formula (II), and is the common name for (-)-D-phenylalanyl-L-cysteinyl-L-phenylalanyl-D-tryptophyl-L-lysyl-L-threonyl-N-[(1R,2R)-2-hydroxy-1-(hydroxymethyl)propyl]-L-cysteinamide cyclic (2→7) disulfide.

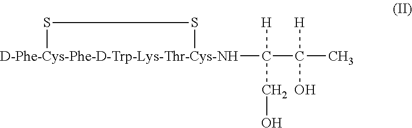

Pasireotide is the common name for cyclo[L-phenyl Gly-D-Trp-L-Lys-O-benzyl-L-Tyr-L-Phe-[(4R)-4-(2-aminoethylcarbamoyloxy)-L-Pro-]].

Lanreotide is the common name for 3-(2-naphthyl)-D-alanyl-L-cysteinyl-L-tyrosyl-D-tryptophyl-L-lysyl-L-valyl-L-cysteinyl-L-threoninamide cyclic(2→7)-disulfide.

The somatostatin analogue may be a free compound, or may form a salt with a pharmaceutically acceptable acid. Examples of such acids include hydrochloric acid, hydrobromic acid, nitric acid, sulfuric acid, phosphoric acid, and other inorganic acids; and methanesulfonic acid, p-toluenesulfonic acid, acetic acid, citric acid, tartaric acid, maleic acid, fumaric acid, malic acid, lactic acid, pamoic acid, and other organic acids. The salt of the somatostatin analogue is preferably hydrochloride or acetate. Examples thereof include octreotide acetate, pasireotide hydrochloride or acetate, lanreotide acetate, and vapreotide acetate.

In addition to the above forms including a free compound and a salt, the somatostatin analogue may be in the form of a microsphere formulation, a gel formulation, an embeddable agent, or the like. With such forms, the stability of the somatostatin analogue can be maintained and the release rate can be controlled, thereby maintaining effective blood concentration of somatostatin analogue at a constant level for a long period of time.

Examples of the microsphere formulation include a particle in which a somatostatin analogue is included in the matrix of a biodegradable polymer. The microsphere formulation may be produced by a known method. Examples include phase separation, spray drying, in-liquid drying, and solvent diffusion.

Examples of biodegradable polymers include polylactic acids, polyglycolic acids, polycaprolactones, polycarbonates, polyester amides, polyanhydrides, polyamino acids, polyorthoesters, polycyanoacrylates, poly(p-dioxanone)s, poly(alkylene oxalate)s, biodegradable polyurethanes, mixtures thereof, and copolymers thereof. If the polymers contain asymmetric carbon atoms, monomers constituting the polymers may be any of D-form, L-form, or DL-form. L-form is preferable. Preferable biodegradable polymers are polylactic acids and polylactic acid-polyglycolic acid copolymers.

The weight-average molecular weight (Mw) of the biodegradable polymer is generally in a range of about 10,000 to 200,000, preferably 25,000 to 100,000, particularly preferably 35,000 to 60,000. Weight-average molecular weight may be measured using gel permeation chromatography (GPC).

The content of the somatostatin analogue in the microsphere is generally about 1 to 30 wt %, preferably 2 to 20 wt %, more preferably 3 to 10 wt %.

The mean particle size of the microsphere is generally about 1 to 250 μm, preferably 10 to 200 μm, more preferably 10 to 90 μm. The mean particle size of the microsphere particle is a volume mean diameter, and can be determined using a laser diffraction particle size distribution meter.

U.S. Pat. No. 5,538,739 or other documents may be referred to for the microsphere formulation of a somatostatin analogue, and the production thereof. JP2013-517228A, JP4489186B and JP4162381B, and the like may be referred to for the gel formulation of a somatostatin analogue, and the production thereof.

### 3. Pharmaceutically Acceptable Carrier for Injection

The pharmaceutically acceptable carrier for injection is used for the preparation of an aqueous suspension or a gel of the active ingredients, i.e., the particle containing tolvaptan or a prodrug thereof and the somatostatin analogue. The carrier for injection generally comprises (a) a suspending agent and/or a wetting agent, (b) optionally, a tonicity agent and/or a bulking agent, (c) optionally, a buffer, (d) optionally, a pH-adjusting agent, (e) optionally, a viscosity-enhancing agent, and (f) optionally, a preservative.

The (a) suspending agent and/or wetting agent are indispensable for suspending the particle containing tolvaptan or a prodrug thereof, and/or the particle containing a somatostatin analogue in water.

Examples of suitable suspending agents include sodium carboxymethyl cellulose, hydroxypropyl cellulose, carboxymethyl cellulose, hydroxypropylethyl cellulose, methylcellulose, hydroxypropylmethyl cellulose, polyvinyl alcohol, macrogol, polyvinylpyrrolidone, and the like. They may be used singly, or in a combination of two or more. In particular, sodium carboxymethyl cellulose and polyvinylpyrrolidone are preferable.

The amount of the suspending agent to be contained is within the range of generally about 0.05 to about 10 w/v %, preferably about 0.1 to about 5 w/v %, based on the total volume of the aqueous suspension or the gel (containing water for injection).

Examples of suitable wetting agents include various surfactants (including nonionic and ionic surfactants), such as gelatin, lecithin (phosphatides), sorbitan esters, polyoxyethylene alkyl ethers, polyoxyethylene castor oil derivatives, polyoxyethylene sorbitan fatty acid esters (e.g., commercially available Tweens®; for example, Tween 20® and Tween (registered trademark, polysorbate 80) (ICI Specialty Chemicals)), poloxamers (e.g., Pluronic F-68® and Pluronic F-108®, which are block copolymers of ethylene oxide and propylene oxide); and poloxamines (e.g., Tetronic 908®, also known as Poloxamine 908® which is a tetrafunctional block copolymer derived from sequential addition of propylene oxide and ethylene oxide to ethylenediamine (BASF Wyandotte Corporation; Parsippany, N.J.)). They may be used singly, or in a combination of two or more. In particular, polysorbate 80 and poloxamers are preferable.

The amount of the wetting agent to be contained is generally about 0.01 to about 5 w/v %, preferably about 0.05 to about 1 w/v %, based on the total volume of the aqueous suspension or the gel.

The carrier for injection may contain (b) a tonicity agent and/or a bulking agent, if necessary. Examples of tonicity agents include sodium chloride, potassium chloride, mannitol, sucrose, lactose, maltose, xylitol, glucose, sorbitol, and the like. They may be used singly, or in a combination of two or more. When the formulation of the present invention is a freeze-dried formulation, the tonicity agent may serve as a bulking agent.

When the injection carrier contains a tonicity agent and/or a bulking agent, the amount of the tonicity agent and/or the bulking agent is generally in a range of about 0.2 to about 12 w/v %, preferably about 0.5 to about 10 w/v %, based on the total volume of the aqueous suspension or the gel.

The carrier for injection may comprise (c) a buffer, if necessary. Examples of buffers suitable in the present invention include sodium citrate, sodium tartrate, sodium phosphate, potassium phosphate, Tris buffer, and the like. They may be used singly, or in a combination of two or more. In particular, sodium phosphate (in particular, sodium dihydrogen phosphate) is preferable.

When the carrier for injection comprises a buffer, the amount of the buffer to be contained is an amount sufficient to adjust the pH of the aqueous suspension or the gel prepared at the time of use to generally about 6 to about 8, and preferably about 7. To achieve such a pH, the buffer, depending on the type, is generally used in an amount within the range of about 0.02 to about 2% by weight, preferably about 0.03 to about 1% by weight, and more preferably about 0.1% by weight, based on the total weight of the aqueous suspension or the gel.

The carrier for injection may comprise (d) a pH-adjusting agent, if necessary. The pH-adjusting agent is used in an amount sufficient to adjust the pH of the aqueous suspension or the gel prepared at the time of use within the range of about 6 to about 8, and preferably about 7; and may be a base or acid depending upon whether the pH of the aqueous suspension or the gel of tolvaptan must be raised or lowered to adjust the pH to the desired neutral pH of about 7. Thus, when the pH must be lowered, an acidic pH-adjusting agent (such as hydrochloric acid, phosphoric acid, and acetic acid, preferably hydrochloric acid) may be used. When the pH must be raised, a basic pH-adjusting agent (such as sodium hydroxide, potassium hydroxide, calcium carbonate, magnesium oxide, and magnesium hydroxide, preferably sodium hydroxide) may be used.

The carrier for injection may comprise (e) a viscosity-enhancing agent, if necessary. Examples of viscosity-enhancing agents include sodium carboxymethyl cellulose, and the like.

The carrier for injection may comprise (f) a preservative, if necessary. Examples of preservatives include quaternary ammonium salts, such as benzylalcohol, benzalkonium chloride and benzethonium chloride; cationic compounds, such as chlorhexidine gluconate; p-hydroxybenzoates, such as methyl parahydroxybenzoate, ethyl parahydroxybenzoate, and propyl parahydroxybenzoate; alcohol compounds such as chlorobutanol and benzyl alcohol; sodium dehydroacetate; thimerosal; and the like.

### 4. Water for Injection

The water for injection is sterile and pyrogen-free water that can be used for production of an injection solution or for dissolution of an injectable medicinal product. The water for injection may be used as it is, or as a solution generally used for injection (liquid for reconstitution or a diluent), such as an aqueous glucose solution or an aqueous sodium chloride solution.

### 5. Injectable Depot Formulation

The drug for preventing and/or treating polycystic kidney disease of the present invention is an injectable depot formulation comprising a particle containing tolvaptan or a prodrug thereof, and a somatostatin analogue.

The form of the formulation of the present invention encompasses a mixed drug (mixture) obtained by mixing a particle containing tolvaptan or a prodrug thereof and a somatostatin analogue, and a drug for combination administering, as separate formulations, an injectable depot formulation comprising a particle containing tolvaptan or a prodrug thereof (hereinafter may be referred to as “Injectable Depot Formulation A”) and an injectable depot formulation containing a somatostatin analogue (hereinafter may be referred to as “Injectable Depot Formulation B”).

For both the mixed drug (mixture) and the drug for combination use, the particle containing tolvaptan may be produced as follows.

The particle containing amorphous tolvaptan may be prepared, for example, by dissolving tolvaptan, and, as necessary, a water-soluble polymer and/or a biodegradable polymer in an organic solvent, and then distilling the organic solvent off to obtain powder. An organic solvent that is capable of dissolving each ingredient and being easily distilled off is selected. Examples of organic solvent include methylene chloride, and a mixed solvent of methylene chloride and alcohol (methanol or ethanol). A particle with a desired particle size distribution can be produced by spray-drying the obtained solution. The process for producing the particle can be performed, for example, according to the process described in JP4210355B.

A particle containing crystalline tolvaptan (in particular, a particle consisting of crystalline tolvaptan) can be prepared, for example, by recrystallizing tolvaptan, and pulverizing the recrystallized tolvaptan into powder. The process for producing the particle can be performed, for example, using a commonly used dry mill (jet mill, hammer mill, or the like) or wet mill (bead mill, or the like). Further, a particle containing crystalline tolvaptan and other ingredients can be obtained, for example, by dissolving other ingredients in a solvent that hardly dissolves crystalline tolvaptan particle, suspending a crystalline tolvaptan particle in the obtained solution, and either spray-drying or spray-freeze-drying the suspension, or pulverizing the freeze-dried cake.

The mixed drug (mixture) encompasses any of solid formulations (powders, cakes, granules, etc.) containing no water for injection, and an aqueous suspension or a gel containing water for injection. An aqueous suspension or a gel may be prepared by mixing a particle containing tolvaptan or a prodrug thereof and a somatostatin analogue with a pharmaceutically acceptable carrier for injection and water for injection.

More specifically, an aqueous suspension may be prepared by filling a container with a particle containing tolvaptan or a prodrug thereof and a particle containing a somatostatin analogue, and then adding a water-containing injection carrier at the time of use. Another possible method is a method of first preparing an aqueous suspension by adding water and a suspending agent or the like to a particle containing tolvaptan or a prodrug thereof, freeze-drying the aqueous suspension to produce a freeze-dried product, adding a particle containing a somatostatin analogue to the freeze-dried product of a particle containing tolvaptan or a prodrug thereof, and adding water or a water-containing injection carrier at the time of use, thereby preparing an aqueous suspension. Another possible method is a method of first preparing an aqueous suspension by adding water and a suspending agent or the like to a particle containing tolvaptan or a prodrug thereof and a particle containing a somatostatin analogue, freeze-drying the aqueous suspension to produce a freeze-dried product, and adding water or a water-containing injection carrier at the time of use, thereby preparing an aqueous suspension.

When the above preparation methods are adopted, the injectable depot formulation of the present invention may be provided, for example, in the form of a kit comprising a container having a particle containing tolvaptan or a prodrug thereof and a somatostatin analogue, and a container having a pharmaceutically acceptable carrier for injection and water for injection; or a kit comprising a container having a particle containing tolvaptan or a prodrug thereof, a somatostatin analogue, and a pharmaceutically acceptable carrier for injection, and a container having water for injection.

The drug for combination use comprises Injectable Depot Formulation A and Injectable Depot Formulation B, as separate formulations.

Injectable Depot Formulation A encompasses any of solid formulations (powders, cakes, granules, etc.) containing no water for injection, and an aqueous suspension containing water for injection. An aqueous suspension may be prepared by mixing a particle containing tolvaptan or a prodrug thereof, a pharmaceutically acceptable carrier for injection, and water for injection.

Further, Injectable Depot Formulation A may also be prepared by mixing a particle containing tolvaptan or a prodrug thereof, and an aqueous solution containing a pharmaceutically acceptable injection carrier and water for injection. More specifically, for example, an aqueous solution containing a pharmaceutically acceptable injection carrier and water for injection is mixed with a sterile particle containing tolvaptan or a prodrug thereof enclosed in a container, such as a vial; the mixture thus obtained is subsequently, for example, shaken vigorously, stirred with a vortex mixer, or subjected to ultrasonic irradiation, thereby preparing a homogeneous aqueous suspension. A homogeneous aqueous suspension can also be prepared by providing two sterile syringes, enclosing a sterile particle containing tolvaptan or a prodrug thereof in one of the syringes, filling an aqueous solution containing a pharmaceutically acceptable injection carrier and water for injection in the other syringe, connecting the two syringes by a connector, and performing pumping repeatedly. An aqueous suspension can be prepared at the time of use using any of the above processes.

When the above preparation methods are adopted, Injectable Depot Formulation A of the present invention may be provided, for example, in the form of a kit comprising a container having a particle containing tolvaptan or a prodrug thereof, and a container having a pharmaceutically acceptable carrier for injection and water for injection.

Other examples include a method of preparing an aqueous suspension by adding water for injection to a solid formulation that does not contain water for injection (i.e., a solid formulation comprising a particle containing tolvaptan or a prodrug thereof and a pharmaceutically acceptable carrier for injection) at the time of use. More specifically, an aqueous suspension comprising a particle containing tolvaptan or a prodrug thereof and a pharmaceutically acceptable carrier for injection is first prepared, and the obtained aqueous suspension is freeze-dried to obtain a freeze-dried product. The freeze-dried product is processed into an aqueous suspension at the time of use using water for injection. As such, this freeze-dried product is suitable as a formulation that can be prepared at the time of use. The characteristics of the particle containing tolvaptan or a prodrug thereof in the suspension before freeze-drying or in the freeze-dried product are maintained also in the aqueous suspension prepared (reconstituted) using water for injection. The preparation of an aqueous suspension from the freeze-dried product may be performed by a method of mixing water for injection with a sterile freeze-dried product enclosed in a container, such as a vial, and then either shaking the mixture vigorously, stirring the suspension liquid with a vortex mixer, or subjecting to ultrasonic irradiation, thereby preparing a homogeneous aqueous suspension.

When the above preparation methods are adopted, Injectable Depot Formulation A of the present invention may be provided, for example, in the form of a kit comprising a container having a particle containing tolvaptan or a prodrug thereof and a pharmaceutically acceptable carrier for injection (i.e., the above freeze-dried product), and a container having water for injection.

Injectable Depot Formulation B encompasses any of solid formulations (powders, cakes, granules, etc.) containing no water for injection, and an aqueous suspension or a gel containing water for injection. An aqueous suspension or a gel may be prepared by mixing a somatostatin analogue, a pharmaceutically acceptable carrier for injection, and water for injection.

The aqueous suspension may be prepared by mixing a particle, such as a microsphere, containing a somatostatin analogue with an aqueous solution containing a pharmaceutically acceptable carrier for injection and water for injection at the time of use. For example, preparation of an aqueous suspension using a microsphere formulation may be performed according to the method disclosed in U.S. Pat. No. 5,538,739 (JPH08-32624B) or the like.

A gel may be prepared, for example, according to the method disclosed in JP2013-517228A, the methods disclosed in Japanese Patent No. 4489186 and No. 4162381.

Examples of Injectable Depot Formulation B include a depot formulation comprising octreotide diacetate (Sandostatin LAR I.M. Injection; Novartis Pharma K.K.), and a depot formulation comprising lanreotide acetate (Somatuline S.C. Injection; Teijin Pharma Limited, Somatuline Depot; Ipsen Pharma Biotech).

Injectable Depot Formulation B of the present invention may be provided in the form of a kit comprising, for example, a container having a particle containing a somatostatin analogue, and a container having water for injection, and, as necessary, a pharmaceutically acceptable carrier for injection; a kit comprising a container having a particle containing a somatostatin analogue, and, as necessary, a pharmaceutically acceptable carrier for injection, and a container having water for injection; or a gel formulation containing a somatostatin analogue.

The drug for preventing and/or treating polycystic kidney disease of the present invention may be provided in the form of a kit (or a package) comprising a container (including a kit) having Injectable Depot Formulation A, and a container (including a kit) having Injectable Depot Formulation B.

A typical prescription example of a drug for combination use comprises, for example, as Injectable Depot Formulation A, an aqueous suspension containing crystalline tolvaptan, a pharmaceutically acceptable carrier for injection, and water for injection, preferably an aqueous suspension containing crystalline tolvaptan consisting essentially of S-tolvaptan, a pharmaceutically acceptable carrier for injection, and water for injection; and, as Injectable Depot Formulation B, an aqueous suspension containing a particle containing a somatostatin analogue, a pharmaceutically acceptable carrier for injection, and water for injection, or a gel containing a somatostatin analogue, water for injection and, if necessary, a pharmaceutically acceptable carrier for injection.

### 6. Administration Method and Dose

Both for the mixed drug (mixture) and the drug for combination use, the injectable depot formulation of the present invention has an injectable form, and is administered intramuscularly or subcutaneously to a patient in need of prevention and/or treatment of polycystic kidney disease, as an aqueous suspension or a gel. The formulation is preferably in the form of an aqueous suspension.

As a mixed drug, an injectable depot formulation obtained by mixing a particle containing tolvaptan or a prodrug thereof with a somatostatin analogue is administered in the form of an aqueous suspension or a gel intramuscularly or subcutaneously to a patient. As a drug for combination use, Injectable depot formulations A and B are administered individually in the form of an aqueous suspension or a gel intramuscularly or subcutaneously at the same time or with a time difference.

The amount (concentration) of tolvaptan or a prodrug thereof to be contained in the injectable depot formulation in the form of an aqueous suspension or a gel is not particularly limited insofar as it is a therapeutically effective amount. “A therapeutically effective amount” means an amount that clinically ensures alleviation of the symptom when a combination of tolvaptan or a prodrug thereof and a somatostatin analogue is administered (both for the mixed drug and the drug for combination use). Further, the amount is preferably determined so that the undesirable effect (for example, pollakiuria etc.) of tolvaptan or a prodrug thereof is reduced or inhibited.

For a mixed drug (mixture), the amount of tolvaptan or a prodrug thereof to be contained in the aqueous suspension or the gel is preferably 50 mg/mL to 500 mg/mL, more preferably 100 mg/mL to 300 mg/mL. A single dose of the aqueous suspension or the gel to be administered intramuscularly or subcutaneously to a patient is generally 0.5 mL to 6 mL, preferably 1 mL to 3 mL.

The amount (concentration) of a somatostatin analogue in the aqueous suspension or the gel is preferably in the range of 1 mg/mL to 300 mg/mL, more preferably in the range of 2 mg/mL to 200 mg/mL. A single dose of the aqueous suspension or the gel to be administered intramuscularly or subcutaneously to a patient is generally 0.5 mL to 6 mL, preferably 1 mL to 3 mL.

For the drug for combination use, the amount of tolvaptan or a prodrug thereof to be contained in the Injectable Depot Formulation A in the form of an aqueous suspension is preferably 50 mg/mL to 500 mg/mL, more preferably 100 mg/mL to 300 mg/mL. A single dose of the aqueous suspension to be administered intramuscularly or subcutaneously to a patient is generally 0.5 mL to 6 mL, preferably 1 mL to 3 mL.

The amount (concentration) of a somatostatin analogue in the Injectable Depot Formulation B in the form of an aqueous suspension or a gel is preferably 1 mg/mL to 300 mg/mL, more preferably 2 mg/mL to 200 mg/mL. A single dose of the aqueous suspension or the gel to be administered intramuscularly or subcutaneously to a patient is generally 0.5 mL to 6 mL, preferably 1 mL to 3 mL.

Both for the mixed drug (mixture) and the drug for combination use, the actual dose is suitably selected according to the dosage regimen, the patient's age and sex, the severity of the disease, and other conditions.

The injectable depot formulation of the present invention is capable of maintaining the therapeutically effective blood concentrations of tolvaptan and somatostatin analogue for a long period of time. Therefore, it is possible to prevent and/or treat polycystic kidney disease while suppressing diuretic activity by intramuscularly or subcutaneously administering the injectable depot formulation, preferably once every two weeks or more (in one or multiple administrations), more preferably once every one month or more (in one or multiple administrations).

A single dose of tolvaptan or a prodrug thereof is an amount equal to about 50 mg to 6000 mg of tolvaptan, and a single dose of a somatostatin analogue is about 2 mg to 1200 mg.

More specifically, when the drug is administered every two weeks, tolvaptan or a prodrug thereof is preferably administered at a tolvaptan dose of 50 to 1000 mg in one or two administrations; and when the drug is administered every four weeks, tolvaptan or a prodrug thereof is preferably administered at a tolvaptan dose of 100 mg to 2000 mg in one or two to four administrations. When the drug is administered every eight weeks, tolvaptan or a prodrug thereof is preferably administered at a tolvaptan dose of 200 to 4000 mg in one or two to four administrations; and when the drug is administered every twelve weeks, tolvaptan or a prodrug thereof is preferably administered at a tolvaptan dose of 300 to 6000 mg in one or two to four administrations.

For example, when the drug is administered every two weeks, a somatostatin analogue is preferably administered at a dose of 2 mg to 200 mg in one or two administrations; and when the drug is administered every four weeks, a somatostatin analogue is preferably administered at a dose of 4 mg to 400 mg in one or two to four administrations. When the drug is administered every eight weeks, a somatostatin analogue is preferably administered at a dose of 8 mg to 800 mg in one or two to four administrations. When the drug is administered every 12 weeks, a somatostatin analogue is preferably administered at a dose of 12 mg to 1200 mg in one or two to four administrations.

For the formulation of the present invention, a particle containing crystalline tolvaptan is preferable because a therapeutically effective blood concentration of tolvaptan can be maintained for a long period of time when the formulation is administered at a long interval. Further, a particle containing crystalline and optically active tolvaptan (in particular, S-tolvaptan) is more preferable because such a particle has high metabolic stability in humans, and thus the effective blood concentration can be kept constant.

### EXAMPLES

The present invention will now be illustrated with the following examples. However, the invention is not limited thereto or thereby.

### Production Example 1

### 0.1% Racemic Tolvaptan SD Powder-Containing Feed

Racemic tolvaptan spray-dried (SD) powder was produced according to the method disclosed in WO2008/156217.

1.5 g of a racemic tolvaptan SD powder formulation (containing 1.0 g of racemic tolvaptan) and 998.5 g of MF powder feed (Oriental Yeast Co., Ltd.) were mixed by shaking in a vinyl bag, thereby preparing a 0.1% racemic tolvaptan-containing feed.

### Production Example 2

### S-Tolvaptan Sustained-Release Formulation

An injectable depot formulation (S-tolvaptan sustained-release formulation) containing a crystalline S-tolvaptan particle was produced as follows.

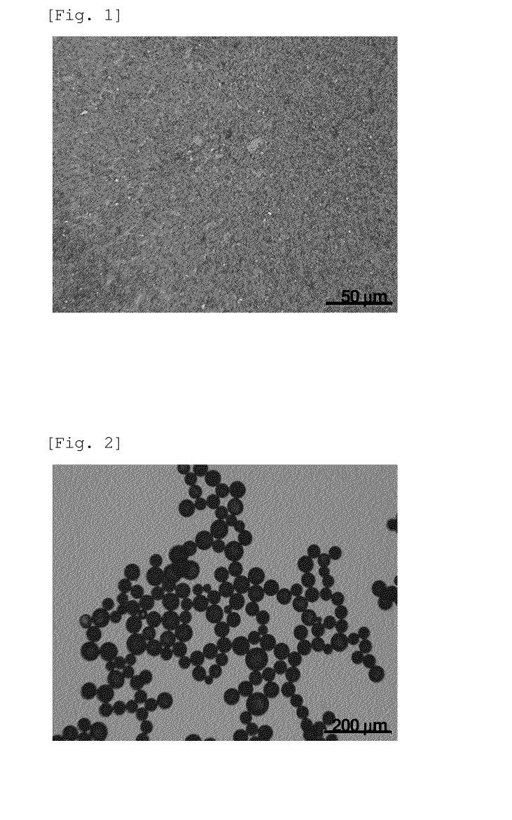

15.0 g of crystalline S-tolvaptan was suspended in 38.0 g of the medium solution shown in Table 1 (equal to a 50 mL formulation). 50 g of zirconia beads having a diameter of 1.5 mm were added to the suspension, and the mixture in the container was stirred to perform bead milling (wet milling), thereby preparing an S-tolvaptan sustained-release formulation. The mean particle size of the crystalline S-tolvaptan particle measured during ultrasonic irradiation using a particle size distribution meter (SALD-3000J, Shimadzu Corporation) was 3.0 μm. FIG. 1 shows a polarizing microscope image of the particle.

TABLE 1
Composition of medium solution

| | Prescribed amount | |
|---|---|---|
| Sodium carboxymethyl cellulose | 6.0 | mg |
| Povidone K17 | 3.0 | mg |
| D-mannitol | 35.0 | mg |
| Sodium dihydrogen phosphate | 0.9 | mg |
| monohydrate | | |
| Sodium hydroxide | q.s. to pH 7.0 | |
| Water for injection | q.s. | |
| Total weight | 0.76 | g |

### Production Example 3

### R-Tolvaptan Sustained-Release Formulation

30.0 g of crystalline R-tolvaptan was suspended in 76.0 g of the medium solution shown in Table 1 (equal to a 100 mL formulation). 150 g of zirconia beads having a diameter of 1.5 mm were added to the suspension, and the mixture in the container was stirred to perform bead milling (wet milling), thereby preparing an R-tolvaptan sustained-release formulation. The mean particle size of the crystalline R-tolvaptan particle measured during ultrasonic irradiation using a particle size distribution meter (SALD-3000J, Shimadzu Corporation) was 1.9 μm. FIG. 3 shows a polarizing microscope image of the particle.

Table 2 shows the prescriptions of the formulations prepared in Production Examples 2 and 3.

TABLE 2
Compositions of Production Example 2 and Production
Example 3 (per mL of formulation)

| | Production | | Production | |
|---|---|---|---|---|
| | Example 2 | | Example 3 | |
| | S-tolvaptan | | R-tolvaptan | |
| | sustained- | | sustained- | |
| | release | | release | |
| | formulation | | formulation | |
| Tolvaptan (anhydrous) | 300 | mg | 300 | mg |
| Sodium carboxymethyl cellulose | 6.0 | mg | 6.0 | mg |
| Povidone K17 | 3.0 | mg | 3.0 | mg |
| D-mannitol | 35.0 | mg | 35.0 | mg |
| Sodium dihydrogen phosphate | 0.9 | mg | 0.9 | mg |
| monohydrate | | | | |
| Sodium hydroxide | q.s. to pH 7.0 | | q.s. to pH 7.0 | |
| Water for injection | q.s. to 1 mL | | q.s. to 1 mL | |
| Total weight | 1.06 | g | 1.06 | g |

### Production Example 4

### Mixed Drug of Crystalline S-Tolvaptan Particle and Sandostatin LAR

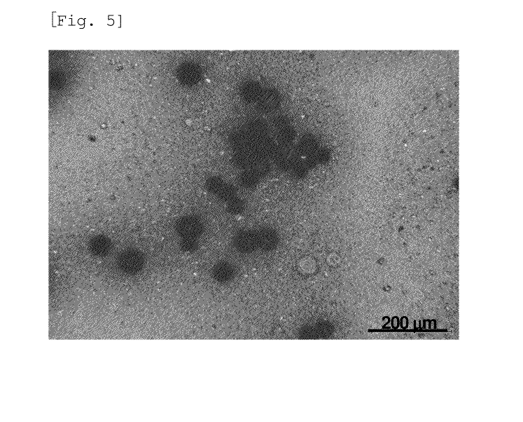

20 mg of Sandostatin LAR intramuscular injection (Novartis Pharma K.K.; see Table 3) was suspended in 1.0 mL of the medium solution shown in Table 1 at an octreotide concentration of 20 mg/mL. 0.5 mL of the resulting suspension was mixed well with 0.5 mL of a formulation of crystalline S-tolvaptan particle prepared in the same manner as in Production Example 2, thereby preparing a mixture of a crystalline S-tolvaptan particle and Sandostatin LAR. FIG. 4 shows a polarizing microscope image of the mixture.

TABLE 3
Formulation Prescription of 20 mg of Sandostatin
LAR Intramuscular Injection

| | Prescribed amount | |
|---|---|---|
| Octreotide acetate | 22.4 | mg*¹ |
| D,L-lactic and glycolic acids | 377.6 | mg |
| copolymer | | |
| D-mannitol | 81.9 | mg |

*¹containing 20 mg octreotide

Table 4 shows the prescription of the formulation prepared in Production Example 4.

TABLE 4
Mixture of crystalline S-tolvaptan particle
and Sandostatin LAR (per mL of formulation)

| | Prescribed amount | |
|---|---|---|
| S-tolvaptan | 150 | mg |
| Octreotide acetate | 11.2 | mg*² |
| D,L-lactic and glycolic acids | 188.8 | mg |
| copolymer | | |
| Sodium carboxymethyl cellulose | 6.0 | mg |
| Povidone K17 | 3.0 | mg |
| D-mannitol | 75.95 | mg |
| Sodium dihydrogen phosphate | 0.9 | mg |
| monohydrate | | |

| | |
|---|---|
| Sodium hydroxide | q.s. to pH 7.0 |
| Water for injection | q.s. |

*²containing 10 mg octreotide

### Production Example 5

### Mixed Drug of Crystalline R-Tolvaptan Particle and Sandostatin LAR

20 mg of Sandostatin LAR intramuscular injection was suspended in 1.0 mL of the medium solution shown in Table 1 at an octreotide concentration of 20 mg/mL. 0.5 mL of the resulting suspension was mixed well with 0.5 mL of a formulation of crystalline R-tolvaptan particle prepared in the same manner as in Production Example 3, thereby preparing a mixture of a crystalline R-tolvaptan particle and Sandostatin LAR. FIG. 5 shows a polarizing microscope image of the mixture.

Table 5 shows the prescription of the formulation prepared in Production Example 5.

TABLE 5
Mixture of crystalline R-tolvaptan particle
and Sandostatin LAR (per mL of formulation)

| | Prescribed amount | |
|---|---|---|
| R-tolvaptan | 150 | mg |
| Octreotide acetate | 11.2 | mg*² |
| D,L-lactic and glycolic acids | 188.8 | mg |
| copolymer | | |
| Sodium carboxymethyl cellulose | 6.0 | mg |
| Povidone K17 | 3.0 | mg |
| D-mannitol | 75.95 | mg |
| Sodium dihydrogen phosphate | 0.9 | mg |
| monohydrate | | |

| | |
|---|---|
| Sodium hydroxide | q.s. to pH 7.0 |
| Water for injection | q.s. |

*²containing 10 mg octreotide

### Production Example 6

### Octreotide Acetate Sustained-Release Formulation

A dedicated diluent (containing water for injection and, as additives, 10 mg of carmellose sodium and 12 mg of D-mannitol per 2 mL) was added to an octreotide acetate sustained-release formulation (powdery microsphere formulation, Sandostatin LAR) to prepare a suspension at an octreotide concentration of 5 mg/mL.

### Production Example 7

### Lanreotide Acetate Sustained-Release Formulation

60 mg of a Somatuline subcutaneous injection (Teijin Pharma Limited) was used as the lanreotide acetate sustained-release formulation. The formulation is a gel formulation, and 244 mg of the gel formulation contains 71.5 mg of lanreotide acetate (containing 60 mg lanreotide).

### Example 1

Individual or combined effects of tolvaptan, which is a vasopressin V2 receptor (V2R) antagonist, and octreotide, which is a somatostatin analogue, against polycystic kidney disease were evaluated using pcy mice, which are PKD model animals.

The pcy mice are adult polycystic kidney disease model mice, and the mode of inheritance is autosomal recessive. In DBA/2FG-pcy mice generated by introducing the pcy gene into DBA/2 mice, cysts were observed with the naked eye from the fourth week, and the kidney volume increased over time until the 30th week. It has been reported that compared to wild-type mice, pcy mice have increased renal cAMP levels and elevated renal mRNA levels of aquaporin-2 and vasopressin V2 receptor (V2R). For details, see Non-Patent Literature 1.

Based on the body weight and the left kidney volume measured by MRI at 4 weeks of age, the pcy mice (male) were divided into the following five groups (each group: 9 mice):

(1) a control group;
(2) a group receiving a 0.1% tolvaptan-containing feed (Production Example 1);
(3) a group receiving an S-tolvaptan sustained-release formulation (Production Example 2) alone (300 mg/kg sc);
(4) a group receiving an octreotide acetate sustained-release formulation (Sandostatin LAR) (Production Example 6) alone (1 mg/body sc); and

(5) a group receiving a combination of an S-tolvaptan sustained-release formulation (Production Example 2) (300 mg/kg sc) and an octreotide acetate sustained-release formulation (Production Example 6) (1 mg/body sc).

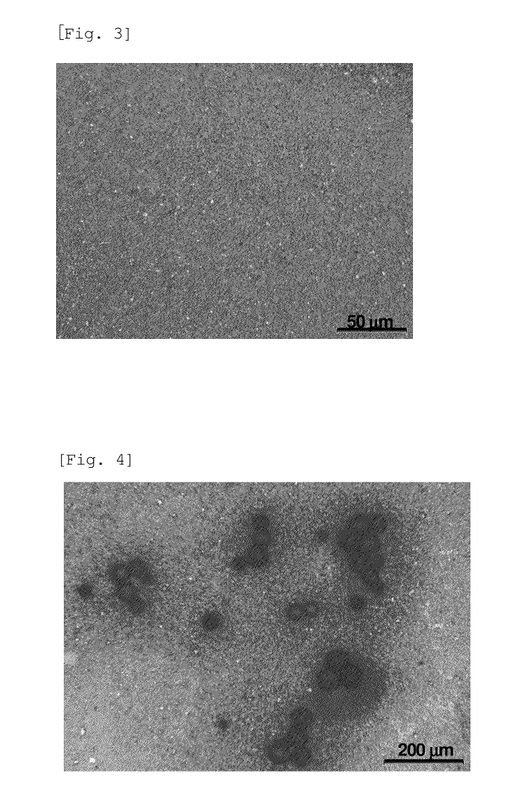

MF feed was given to all of the groups during the test. FIG. 2 shows a microscope image of Sandostatin LAR.

In the group receiving an S-tolvaptan sustained-release formulation alone and the group receiving a combination of S-tolvaptan and an octreotide acetate, an S-tolvaptan formulation (60 mg/mL) obtained by diluting the S-tolvaptan sustained-release formulation of Production Example 2 with the medium solution shown in Table 1 was administered subcutaneously to mice at 5 and 11 weeks of age in an amount of 300 mg/kg. In the group receiving an octreotide acetate sustained-release formulation alone and the group receiving a combination of an octreotide acetate and S-tolvaptan, an octreotide acetate sustained-release formulation at a concentration of 5 mg/mL was administered subcutaneously to mice at 5, 9, and 13 weeks of age in an amount of 1 mg/body.

In each group, the drug treatment was started from 5 weeks of age, and measurement of the left kidney volume for each individual by MRI was performed at 12 weeks of age. Urine was collected from each mouse at 15 weeks of age using metabolic cages for 19 hours, and urine volume was measured.

Table 6 shows change in the left kidney volume (A mm³) calculated from the left kidney volumes measured by MRI at 4 weeks of age (at the time of grouping) and at 12 weeks of age (after 7 weeks of testing). The left kidney volume of the pcy control mouse at 4 weeks of age was 250.8±8.2 mm³, and the kidney volume increased 4-fold or more at 12 weeks of age. The increase in kidney volume was significantly suppressed in the group receiving a 0.1% tolvaptan-containing feed, compared with the control group; however, the significant suppression of kidney volume was not observed in the group receiving an S-tolvaptan sustained-release formulation alone or in the group receiving an octreotide acetate sustained-release formulation alone.

In contrast, significant suppression of kidney volume was observed in the group receiving the two drugs, compared with the pcy control group (p<0.01), and the effect was in the same level as the group receiving a 0.1% tolvaptan-containing feed. Further, in the group receiving both the S-tolvaptan sustained-release formulation and the octreotide acetate sustained-release formulation, significant suppression of kidney volume was observed, compared with the group receiving either drug alone (p<0.05).

For the group receiving a 0.1% tolvaptan-containing feed and the group receiving a combination of an S-tolvaptan sustained-release formulation and an octreotide acetate sustained-release formulation, the plasma concentration of tolvaptan was 254 ng/mL and 38.5 ng/mL, respectively, at 11 weeks of age. The group receiving a combination of an S-tolvaptan sustained-release formulation and an octreotide acetate sustained-release formulation showed the same level of significant suppression of kidney volume at a remarkably low plasma concentration of tolvaptan, compared with the group receiving a 0.1% tolvaptan-containing feed.

TABLE 6
Change in left kidney volume (mm³) of pcy
mice (12 weeks of age-4 weeks of age)

| | | | Δ Left | Results of statistical analysis | |
|---|---|---|---|---|---|
| | | | kidney | | Comparison |
| | | | volume | Comparison | with combination |
| | | Number | (mm³) | with control | administration |
| Group of mice | | of mice | Mean ± SE | group | group |
| 1 | pcy control | 9 | 885.7 ± 98.7 | — | — |
| 2 | pcy receiving | 9 | 517.3 ± 34.3 | P < 0.01 | — |
| | 0.1% tolvaptan- | | | | |
| | containing feed | | | | |
| 3 | pcy receiving | 9 | 686.8 ± 46.6 | NS | P < 0.05 |
| | S-tolvaptan | | | | |
| | sustained-release | | | | |
| | formulation | | | | |
| | (300 mg/kg sc) | | | | |
| 4 | pcy receiving | 9 | 685.6 ± 46.1 | NS | P < 0.05 |
| | octreotide acetate | | | | |
| | sustained-release | | | | |
| | formulation | | | | |
| | (1 mg/body sc) | | | | |
| 5 | pcy receiving | 9 | 535.3 ± 51.5 | P < 0.01 | — |
| | S-tolvaptan | | | | |
| | sustained-release | | | | |
| | formulation | | | | |
| | (300 mg/kg sc) + | | | | |
| | octreotide acetate | | | | |
| | sustained-release | | | | |
| | formulation | | | | |
| | (1 mg/body sc) | | | | |

Each value indicates the mean value ± SEM.
For comparison with the control group, Dunnett's test (two tailed) was used.
For comparison between the single-drug administration group and the combination administration group, the closed testing procedure was used.
NS = not significant

Table 7 shows urine volumes of the pcy control group, as well as the group receiving a 0.1% tolvaptan-containing feed and the group receiving a combination of an S-tolvaptan sustained-release formulation and an octreotide acetate sustained-release formulation, in both of which suppression of the kidney volume was observed. The urine volumes were measured at 15 weeks of age. Although the increase in the kidney volume was significantly suppressed in the group receiving a 0.1% tolvaptan-containing feed, the urine volume of this group was significantly increased compared with the pcy control group. In contrast, the urine volume was significantly decreased in the group receiving a combination of an S-tolvaptan sustained-release formulation and an octreotide acetate sustained-release formulation, compared with the group receiving a 0.1% tolvaptan-containing feed in which the same level of significant suppression of an increase in kidney volume was observed (p<0.05).

TABLE 7
Urine volume (mL) of pcy mice (15 weeks of age)

| | | | | Results of statistical analysis | |
|---|---|---|---|---|---|
| | | | Urine | | Comparison |
| | | | volume | Comparison | with 0.1% tolvaptan |
| | | Number | (mL/19 h) | with control | administration |
| Group of mice | | of mice | Mean ± SE | group | group |
| 1 | pcy control | 9 | 3.72 ± 0.48 | — | — |
| 2 | pcy receiving | 9 | 6.64 ± 0.46 | P < 0.01 | — |
| | 0.1% tolvaptan- | | | | |
| | containing feed | | | | |
| 5 | pcy receiving | 9 | 5.21 ± 0.49 | NS | P < 0.05 |
| | S-tolvaptan | | | | |
| | sustained-release | | | | |
| | formulation | | | | |
| | (300 mg/kg sc) + | | | | |
| | octreotide acetate | | | | |
| | sustained-release | | | | |
| | formulation | | | | |
| | (1 mg/body sc) | | | | |

Each value indicates the mean value ± SEM.
For comparison with the control group, Dunnett's test (two tailed) was used.
For comparison between the combination administration group and the 0.1% tolvaptan administration group, t-test (two tailed) was used.
NS = not significant

The results shown in Table 6 revealed that the increase in cystic kidney was significantly suppressed by the administration of a combination of an S-tolvaptan sustained-release formulation and an octreotide acetate sustained-release formulation in doses that are too low to exhibit the effect of significantly suppressing the kidney volume if either of the drugs is administered alone. More specifically, the results revealed that it is possible to suppress the increase in cystic kidney by the synergistic effect of the combined administration of an S-tolvaptan sustained-release formulation and an octreotide acetate sustained-release formulation.

The results shown in Table 7 revealed that it is possible to suppress an increase in cystic kidney without increasing the urine volume compared with the group receiving a tolvaptan-containing feed by the combined administration of an S-tolvaptan sustained-release formulation and an octreotide acetate sustained-release formulation.

### Example 2

Individual or combined effects of tolvaptan, which is a vasopressin V2 receptor (V2R) antagonist, and lanreotide, which is a somatostatin analogue, on polycystic kidney disease with regard to the kidney function were evaluated using PCK rats, which are PKD model animals.

PCK rats are spontaneous PKD model animals, and have the spontaneously mutated Pkhd1, which is the orthologue of the causative gene in human ARPKD. However, due to the similarity such as mild progress of pathological condition or the fact that male shows more severe pathosis than female, PKD rats are often used for the basic research for the therapeutic intervention of ADPKD. It has been reported that compared to wild-type rats, PCK rats have increased renal cAMP levels and elevated renal mRNA levels of aquaporin-2 and vasopressin V2 receptor. For details, see Non-Patent Literature 1.

Based on the plasma concentration of albumin at 13 weeks of age and the right kidney volume measured by MRI, PCK rats (male) were divided into the following four groups (each group: 11-12 rats):

(1) a control group;
(2) a group receiving an R-tolvaptan sustained-release formulation (Production Example 3) alone (100 mg/kg, i.m.);
(3) a group receiving a lanreotide acetate sustained-release formulation (Somatuline subcutaneous injection, Teijin Pharma Limited) (Production Example 7) alone (2.5 mg/body, s.c.); and
(4) a group receiving a combination of an R-tolvaptan sustained-release formulation (Production Example 3) and a lanreotide acetate sustained-release formulation (Production Example 7).

Crl:CD(SD) was used for the normal control rats (N=5). MF feed was given to all of the groups during the test.

In the group receiving an R-tolvaptan sustained-release formulation alone and the group receiving a combination of R-tolvaptan and a lanreotide acetate, the R-tolvaptan sustained-release formulation of Production Example 3 was intramuscularly administered to the right gastrocnemius muscle of rats at 14, 15, and 20 weeks of age in an amount of 100 mg/kg. In the group receiving a lanreotide acetate sustained-release formulation alone and the group receiving a combination of a lanreotide acetate and R-tolvaptan, a lanreotide acetate sustained-release formulation (0.293 mg/mg gel) was administered subcutaneously to rats at 14 and 19 weeks of age in an amount of 2.5 mg/body.

In each group, the drug treatment was started from 14 weeks of age and ended at 21 weeks of age. Urine was collected from each rat at 20 weeks of age using metabolic cages for 20 hours, and creatinine excretion in urine was measured. Blood was collected from each rat at the final, 21st week of age, and plasma concentration of creatinine (mg/dL) was measured. Based on the creatinine excretion in urine, creatinine clearance (mL/min/100 g) was calculated as an index of kidney function.

Table 8 shows the plasma concentration of creatinine at the end of the test (at 21 weeks of age), and Table 9 shows creatinine clearance. An increase in plasma concentration of creatinine and a decrease in creatinine clearance were observed in PCK control rats, compared with normal SD rats, thus showing decreased kidney function. Although significant variations in plasma concentration of creatinine and in creatinine clearance were not observed by the individual administrations of the R-tolvaptan sustained-release formulation and the lanreotide acetate sustained-release formulation, significant decrease in plasma concentration of creatinine (p<0.05) and significant increase in creatinine clearance (p<0.05) compared with the PCK control group were observed in the group receiving the combination of the R-tolvaptan sustained-release formulation and the lanreotide acetate sustained-release formulation. Further, in the group receiving the combination of the R-tolvaptan sustained-release formulation and the lanreotide acetate sustained-release formulation, significant decrease in plasma concentration of creatinine and significant increase in creatinine clearance (p<0.05) compared with the group receiving either drug alone were observed.

TABLE 8
Plasma concentration of creatinine (mg/dL) of PCK rats (21 weeks of age)

| | | | | Results of statistical analysis | |
|---|---|---|---|---|---|
| | | | Plasma | | Comparison |
| | | | concentration | Comparison | with combination |
| | | Number | of creatinine | with control | administration |
| Group of mice | | of mice | Mean ± SE | group | group |
| 1 | Normal SD | 5 | 0.26 ± 0.02 | — | — |
| 2 | PCK control | 12 | 0.35 ± 0.01 | — | — |
| 3 | PCK receiving | 11 | 0.36 ± 0.01 | NS | P < 0.01 |
| | R-tolvaptan | | | | |
| | sustained-release | | | | |
| | formulation | | | | |
| | (100 mg/kg im) | | | | |
| 4 | PCK receiving | 11 | 0.33 ± 0.01 | NS | P < 0.05 |
| | lanreotide acetate | | | | |
| | sustained-release | | | | |
| | formulation | | | | |
| | (2.5 mg/body sc) | | | | |
| 5 | PCK receiving | 11 | 0.30 ± 0.01 | P < 0.05 | — |
| | R-tolvaptan | | | | |
| | sustained-release | | | | |
| | formulation | | | | |
| | (100 mg/kg im) + | | | | |
| | lanreotide acetate | | | | |
| | sustained-release | | | | |
| | formulation | | | | |
| | (2.5 mg/body sc) | | | | |

Each value indicates the mean value ± SEM.
For comparison with the control group, Dunnett's test (two tailed) was used.
For comparison between the single-drug administration group and the combination administration group, the closed testing procedure was used.
NS = not significant

TABLE 9
Creatinine clearance (mL/min/100 g) of PCK rats (21 weeks of age)

| | | | | Results of statistical analysis | |
|---|---|---|---|---|---|
| | | | | | Comparison with |
| | | | Creatinine | Comparison | combination |
| | | Number | clearance | with control | administration |
| | Group of mice | of mice | Mean ± SE | group | group |
| 1 | Normal SD | 5 | 1.15 ± 0.10 | — | — |
| 2 | PCK control | 12 | 0.66 ± 0.01 | — | — |
| 3 | PCK receiving | 11 | 0.71 ± 0.03 | NS | P < 0.05 |
| | R-tolvaptan | | | | |
| | sustained-release | | | | |
| | formulation | | | | |
| | (100 mg/kg im) | | | | |
| 4 | PCK receiving | 11 | 0.73 ± 0.03 | NS | P < 0.05 |
| | lanreotide acetate | | | | |
| | sustained-release | | | | |
| | formulation | | | | |
| | (2.5 mg/body sc) | | | | |
| 5 | PCK receiving | 11 | 0.84 ± 0.04 | P < 0.05 | — |
| | R-tolvaptan | | | | |
| | sustained-release | | | | |
| | formulation | | | | |
| | (100 mg/kg im) + | | | | |
| | lanreotide acetate | | | | |
| | sustained-release | | | | |
| | formulation | | | | |
| | (2.5 mg/body sc) | | | | |

Each value indicates the mean value ± SEM.
For comparison with the control group, Dunnett's test (two tailed) was used.
For comparison between the single-drug administration group and the combination administration group, the closed testing procedure was used.
NS = not significant

The above results revealed that it is possible to suppress the decrease in the kidney function of PCK rats due to polycystic kidney disease by the synergistic effect of the combined administration of an R-tolvaptan sustained-release formulation and a lanreotide acetate sustained-release formulation.

1. A drug for preventing and/or treating polycystic kidney disease, wherein the drug is an injectable depot formulation comprising a particle containing tolvaptan or a prodrug thereof, and a somatostatin analogue.

2. The drug for preventing and/or treating polycystic kidney disease according to claim 1, wherein the tolvaptan or a prodrug thereof is crystalline.

3. The drug for preventing and/or treating polycystic kidney disease according to claim 1, wherein the tolvaptan or a prodrug thereof is an optically active body.

4. The drug for preventing and/or treating polycystic kidney disease according to claim 3, wherein the optically active tolvaptan or a prodrug thereof is tolvaptan consisting essentially of R-tolvaptan or a prodrug thereof, or tolvaptan consisting essentially of S-tolvaptan or a prodrug thereof.

5. The drug for preventing and/or treating polycystic kidney disease according to claim 1, wherein the content of tolvaptan or a prodrug thereof in the particle containing tolvaptan or a prodrug thereof is 50 to 100 wt %.

6. The drug for preventing and/or treating polycystic kidney disease according to claim 1, wherein the particle containing tolvaptan or a prodrug thereof has a mean particle size of about 1 to 100 μm.

7. The drug for preventing and/or treating polycystic kidney disease according to claim 1, wherein the somatostatin analogue is at least one member selected from the group consisting of somatostatin, octreotide, pasireotide, lanreotide, vapreotide, and their salts.

8. The drug for preventing and/or treating polycystic kidney disease according to claim 1, wherein the somatostatin analogue is a microsphere formulation or a gel formulation.

9. The drug for preventing and/or treating polycystic kidney disease according to claim 1, wherein the injectable depot formulation comprises a mixture of a particle containing tolvaptan or a prodrug thereof and a somatostatin analogue (mixed drug).

10. The drug for preventing and/or treating polycystic kidney disease according to claim 9, wherein the injectable depot formulation contains a pharmaceutically acceptable carrier for injection.

11. The drug for preventing and/or treating polycystic kidney disease according to claim 9, wherein the injectable depot formulation contains water for injection.

12. The drug for preventing and/or treating polycystic kidney disease according to claim 11, wherein the injectable depot formulation is in the form of an aqueous suspension, and the aqueous suspension contains tolvaptan or a prodrug thereof in an amount of 50 mg/mL to 500 mg/mL.

13. The drug for preventing and/or treating polycystic kidney disease according to claim 9, wherein the injectable depot formulation is administered subcutaneously or intramuscularly.

14. The drug for preventing and/or treating polycystic kidney disease according to claim 1, wherein the injectable depot formulation comprises a combination of Injectable Depot Formulation A comprising a particle containing tolvaptan or a prodrug thereof, and Injectable Depot Formulation B comprising a somatostatin analogue, and Formulation A and Formulation B are administered in combination (drug for combination use).

15. The drug for preventing and/or treating polycystic kidney disease according to claim 14, wherein Injectable Depot Formulation A contains a pharmaceutically acceptable carrier for injection.

16. The drug for preventing and/or treating polycystic kidney disease according to claim 14, wherein Injectable Depot Formulation B contains a pharmaceutically acceptable carrier for injection.

17. The drug for preventing and/or treating polycystic kidney disease according to claim 14, wherein Injectable Depot Formulations A and B contain water for injection.

18. The drug for preventing and/or treating polycystic kidney disease according to claim 14, wherein Injectable Depot Formulation A is in the form of an aqueous suspension, and the aqueous suspension contains tolvaptan or a prodrug thereof in an amount of 50 mg/mL to 500 mg/mL.

19. The drug for preventing and/or treating polycystic kidney disease according to claim 14, wherein Injectable Depot Formulation B is in the form of an aqueous suspension or a gel.

20. The drug for preventing and/or treating polycystic kidney disease according to claim 14, wherein each of Injectable Depot Formulations A and B is administered subcutaneously or intramuscularly.

21. The drug for preventing and/or treating polycystic kidney disease according to claim 1, wherein the tolvaptan or a prodrug thereof is tolvaptan.
